(19) United States
(12) Patent Application Publication
(10) Pub. No.: US 2026/0183455 A1
(43) Pub. Date: Jul. 2, 2026

(54) MEDICAL DEVICE AND METHOD FOR MANUFACTURING MEDICAL DEVICE

(71) Applicant: TERUMO KABUSHIKI KAISHA, Tokyo (JP)

(72) Inventor: Nozomu Watanabe, Kanagawa (JP)

(73) Assignee: TERUMO KABUSHIKI KAISHA, Tokyo (JP)

(21) Appl. No.: 19/544,121

(22) Filed: Feb. 19, 2026

Related U.S. Application Data
(63) Continuation of application No. PCT/JP2024/028183, filed on Aug. 7, 2024.

(30) Foreign Application Priority Data
Sep. 28, 2023 (JP) 2023-166805

(51) Int. Cl. A61L 29/08; A61L 29/02; A61L 29/06; A61L 31/02; A61L 31/06; A61L 31/10

(52) U.S. Cl. CPC A61L 29/085; A61L 29/02; A61L 29/06; A61L 31/022; A61L 31/06; A61L 31/10; A61L 2400/10; A61L 2420/00

(57) ABSTRACT

A method for manufacturing a medical device having a surface lubricating layer that exhibits excellent durability even in a heat treatment at a low temperature. The method for manufacturing includes a medical device, the medical device including a base material layer and a surface lubricating layer supported on at least a part of the base material layer, the method including: preparing a coating liquid containing a block copolymer having a structural unit (A) derived from a reactive monomer having an epoxy group and a structural unit (B) derived from a hydrophilic monomer, an isocyanate compound having two or more isocyanate groups, and a solvent; and applying the coating liquid onto the base material layer.

### CROSS-REFERENCES TO RELATED APPLICATIONS

This application is a continuation of International Application No. PCT/JP2024/028183 filed on Aug. 7, 2024, which claims priority to Japanese Application No. 2023-166805 filed on Sep. 28, 2023, the entire content of both of which is incorporated herein by reference.

### TECHNOLOGICAL FIELD

The present disclosure relates to a medical device and a method for manufacturing a medical device.

### BACKGROUND DISCUSSION

Medical devices inserted into a living body such as a catheter, a guide wire, and an indwelling needle are required to exhibit an excellent lubricating property in order to reduce damage to tissues such as a blood vessel and improve operability of an operating surgeon. For this reason, methods for coating a base material layer surface with a hydrophilic polymer having the lubricating property have been developed and put to practical use. In such a medical device, elution and peeling of the hydrophilic polymer from the surface of the base material layer is a problem in terms of safety and maintenance of operability. Therefore, coating with a hydrophilic polymer is required not only to have the excellent lubricating property but also to have durability against loads such as wear and abrasion.

From such a viewpoint, Japanese Patent Application Publication No. 2015-57081 A discloses a medical device in which a water-soluble or water-swellable polymer is dissolved in a solvent in which a base material of the medical device swells to produce a polymer solution, the base material of the medical device is immersed in the polymer solution to swell, and the polymer is crosslinked or polymerized on the surface of the base material to form a surface lubricating layer on the surface of the base material. According to the technique disclosed in Japanese Patent Application Publication No. 2015-57081 A, a surface lubricating layer exhibiting a relatively good lubricating property can be fixed to a base material.

However, in the case of a conventional surface lubricating layer, heating at a high temperature is required in order to obtain high durability. In this case, it is difficult to heat-treat a base material having low heat resistance at a relatively high temperature, and to form a surface lubricating layer having sufficient durability.

Therefore, there is a demand for a technique capable of achieving high durability (particularly, sliding durability) even in a heat treatment at a temperature of less than 100° C. in a medical device having a surface lubricating layer.

### SUMMARY

A method for manufacturing a medical device is disclosed that has a surface lubricating layer that exhibits excellent durability (particularly, sliding durability) even in a heat treatment at a low temperature.

The present inventor has intensively studied to solve the above problems. As a result, the present inventor has found that by combining an isocyanate compound having two or more isocyanate groups with a hydrophilic block copolymer having an epoxy group and applying these two components as a one-liquid system.

The present disclosure has the following configuration and includes the following aspects and embodiments.

An aspect of the present disclosure has the following configuration:

(1) A method for manufacturing a medical device, the medical device including a base material layer and a surface lubricating layer supported on at least a part of the base material layer, the method including: preparing a coating liquid containing a block copolymer having a structural unit (A) derived from a reactive monomer having an epoxy group and a structural unit (B) derived from a hydrophilic monomer, an isocyanate compound having two or more isocyanate groups, and a solvent; and applying the coating liquid onto the base material layer.

(2) In the manufacture method according to (1) above, the coating liquid preferably contains the block copolymer and the isocyanate compound at a mass ratio of 100:0.5 or more and 100:8 or less.

(3) In the manufacture method according to (1) or (2) above, the isocyanate compound is preferably an aliphatic or aromatic isocyanate compound having 6 carbon to 20 carbon atoms.

(4) In the manufacture method according to any one of (1) to (3) above, the isocyanate compound preferably includes at least one selected from the group consisting of hexamethylene diisocyanate, toluene diisocyanate, and 4,4′-diphenylmethane diisocyanate.

(5) In the manufacture method according to any one of (1) to (4) above, it is preferable to further include applying the coating liquid onto the base material layer to form a coating film, and then heat-treating the coating film at a temperature of 80° C. or higher and lower than 100° C. (80° C. to 100° C.) for 1 hour or more and 10 hours or less (1 hour to 10 hours).

(6) In the manufacture method according to any one of (1) to (5) above, the reactive monomer having an epoxy group preferably includes at least one selected from the group consisting of glycidyl acrylate, glycidyl methacrylate, 3,4-epoxycyclohexyl methyl acrylate, 3,4-epoxycyclohexyl methyl methacrylate, β-methyl glycidyl methacrylate, and allyl glycidyl ether.

(7) In the manufacture method according to any one of (1) to (6) above, the hydrophilic monomer preferably includes at least one selected from the group consisting of N,N-dimethylacrylamide, acrylamide, 2-hydroxyethyl methacrylate, and N-vinylpyrrolidone.

(8) In the manufacture method according to any one of (1) to (7) above, the solvent preferably includes at least one selected from the group consisting of tetrahydrofuran, N,N-dimethylformamide, dimethyl sulfoxide, and acetone.

(9) In the manufacture method according to any one of (1) to (8) above, the base material layer preferably contains at least one selected from the group consisting of a metal, a polyamide resin, a polyolefin resin, a polyester resin, and a polyurethane resin.

Another aspect of the present disclosure has the following configuration:

(10) A medical device including: a base material layer; and a surface lubricating layer supported on at least a part of the base material layer, wherein the surface lubricating layer comprises a crosslinking reaction product of an epoxy group of a block copolymer having a structural unit (A) derived from a reactive monomer having an epoxy group and a structural unit (B) derived from a hydrophilic monomer and an isocyanate group of an isocyanate compound having two or more isocyanate groups.

(11) In the medical device according to the above (10) above, the reactive monomer having an epoxy group preferably includes at least one selected from the group consisting of glycidyl acrylate, glycidyl methacrylate, 3,4-epoxycyclohexyl methyl acrylate, 3,4-epoxycyclohexyl methyl methacrylate, β-methyl glycidyl methacrylate, and allyl glycidyl ether.

(12) In the medical device according to any one of (10) or (11) above, the hydrophilic monomer preferably includes at least one selected from the group consisting of N, N-dimethylacrylamide, acrylamide, 2-hydroxyethyl methacrylate, and N-vinylpyrrolidone.

(13) In the medical device according to any one of (10) to (12) above, the isocyanate compound preferably includes at least one selected from the group consisting of hexamethylene diisocyanate, toluene diisocyanate, and 4,4′-diphenylmethane diisocyanate.

(14) In the medical device according to any one of (10) to (13) above, the base material layer preferably contains at least one selected from the group consisting of a metal, a polyamide resin, a polyolefin resin, a polyester resin, and a polyurethane resin.

(15) In accordance with another aspect, a lubricating layer for coating a base material of a medical device, the lubricating layer comprising: a block copolymer having a structural unit (A) derived from a reactive monomer having an epoxy group and a structural unit (B) derived from a hydrophilic monomer, an isocyanate compound having two or more isocyanate groups, and a solvent.

### BRIEF DESCRIPTION OF THE DRAWINGS

FIG. 1 is a partial cross-sectional view schematically illustrating a lamination configuration of a surface of a representative embodiment of a medical device according to the present disclosure.

FIG. 2 is a partial cross-sectional view schematically illustrating a different structure example of the lamination configuration of the surface as an application example of the embodiment of FIG. 1.

FIG. 3 is a graph obtained by evaluating the sliding durability of a surface lubricating layer provided on a stainless steel base material (Example 1 and Comparative Example 1).

FIG. 4 is a graph obtained by evaluating the sliding durability of a surface lubricating layer provided on a nylon elastomer base material (Example 2 and Comparative Example 2).

FIG. 5 is a graph obtained by evaluating the sliding durability of a surface lubricating layer provided on a polyester elastomer base material (Example 3 and Comparative Example 3).

FIG. 6 is a graph obtained by evaluating the sliding durability of a surface lubricating layer provided on a polyethylene base material (Example 4 and Comparative Example 4).

FIG. 7 is a graph obtained by evaluating the sliding durability of a surface lubricating layer provided on a stainless steel base material (Example 1 and Comparative Example 5).

FIG. 8 is Table 1, which shows that a medical device having a surface lubricating layer excellent in sliding durability and sliding property can be obtained by applying a coating liquid containing an isocyanate compound and a block copolymer onto a base material layer.

### DETAILED DESCRIPTION

Set forth below with reference to the accompanying drawings is a detailed description of embodiments of a medical device and a method for manufacturing a medical device. Hereinafter, embodiments of the present disclosure will be described, but present disclosure is not limited only to the following embodiments, and various modifications can be made within the scope of claims. The embodiments described in the present specification can be optionally combined with each other to form another embodiment. That is, an aspect in which two or three or more of preferable individual aspects of the present disclosure described below are combined is also regarded as a preferable aspect of the present disclosure and disclosed in the present specification (that is, it constitutes a legitimate basis for amendment). In the present specification, a range of “X to Y” includes X and Y and indicates “X or more and Y or less”. Unless otherwise specified, operations and measurements of physical properties and the like are performed at room temperature (20° C. to 25° C.) and at relative humidity of 40% to 50% RH. A and/or B refers to A, B or a combination of these.

A method for manufacturing a medical device according to an aspect of the present disclosure is a method for manufacturing a medical device, the medical device including a base material layer and a surface lubricating layer supported on at least a part of the base material layer, the method including: preparing a coating liquid containing a block copolymer having a structural unit (A) derived from a reactive monomer having an epoxy group and a structural unit (B) derived from a hydrophilic monomer, an isocyanate compound having two or more isocyanate groups, and a solvent; and applying the coating liquid onto the base material layer.

According to the method for manufacturing a medical device of the present disclosure, a medical device having a surface lubricating layer that exhibits excellent durability (particularly, sliding durability) even in a heat treatment at a low temperature (for example, lower than 100° C.) can be manufactured.

In the present specification, the structural unit (A) derived from a reactive monomer having an epoxy group is also simply referred to as a “structural unit (A) according to the present disclosure” or “structural unit (A)”. In the present specification, the structural unit (B) derived from a hydrophilic monomer is also simply referred to as a “structural unit (B) according to the present disclosure” or “structural unit (B)”. In the present specification, the block copolymer having the structural units (A) and (B) is also simply referred to as a “block copolymer according to the present disclosure” or “block copolymer”.

In the present specification, the isocyanate compound having two or more isocyanate groups is also simply referred to as an “isocyanate compound according to the present disclosure” or “isocyanate compound”.

In the present specification, when a structural unit is defined to be “derived” from a certain monomer, it means that the structural unit is a divalent structural unit generated by cleavage of one bond of a polymerizable unsaturated double bond of the corresponding monomer.

In the present specification, the term “(meth)acryl” refers to both acryl and methacryl. Therefore, for example, the term “(meth)acrylic acid” refers to both acrylic acid and methacrylic acid. Similarly, the term “(meth)acryloyl” refers to both acryloyl and methacryloyl. Therefore, for example, the term “(meth)acryloyl group” refers to both acryloyl group and methacryloyl group. Further similarly, the term “(meth)acrylate” refers to both acrylate and methacrylate. For example, the term “alkoxyalkyl (meth)acrylate” encompasses both an alkoxyalkyl acrylate and an alkoxyalkyl methacrylate.

In the present disclosure, a coating liquid containing a block copolymer having a structural unit (A) derived from a reactive monomer having an epoxy group and a structural unit (B) derived from a hydrophilic monomer, an isocyanate compound having two or more isocyanate groups, and a solvent is prepared, and the coating liquid is applied onto the base material layer. That is, the present disclosure is characterized by forming a surface lubricating layer using a block copolymer and an isocyanate compound as a one-liquid system.

In the medical device having a surface lubricating layer, a surface lubricating layer having high durability is required. As one means for obtaining a surface lubricating layer having high durability, it is considered to increase the degree of crosslinking of the polymer constituting the surface lubricating layer, and it is considered to use a crosslinking aid during a crosslinking reaction. For example, when a block copolymer having a structural unit derived from a reactive monomer having an epoxy group is subjected to a crosslinking reaction, a method using an acid or a base as a crosslinking aid can be used. However, a crosslinking aid (acid or base) may remain in the formed surface lubricating layer, and further reaction may proceed in the surface lubricating layer. Then, the temporal stability of the surface lubricating layer is deteriorated, and as a result, the durability of the surface lubricating layer is deteriorated. Therefore, when such a crosslinking aid is used, it is necessary to remove the crosslinking aid remaining in the surface lubricating layer after the crosslinking reaction, and the operation becomes complicated. Therefore, as a means for maintaining and improving the durability of the surface lubricating layer, a method of securing the reactivity of the polymer by a heat treatment at a high temperature or prolonging the heating time has been adopted. However, in such a case, there is a problem in that a base material having low heat resistance cannot be used.

Therefore, a method capable of performing a crosslinking reaction at a low temperature was studied. As a result, the present inventor has found that a block copolymer and an isocyanate compound are mixed in one liquid (one-liquid system), and a crosslinking reaction is performed in a state where the epoxy group of the block copolymer and the isocyanate compound are in contact with each other, whereby a surface lubricating layer having high durability can be obtained even in a crosslinking reaction at a low temperature. That is, the present disclosure has found that a surface lubricating layer excellent in durability can be formed by a heat treatment at a low temperature (for example, lower than 100° C.) by using a coating liquid containing a block copolymer and an isocyanate compound as a one-liquid system.

As a mechanism by which such an effect can be exhibited by using a coating liquid containing a block copolymer and an isocyanate compound as a one-liquid system, the following is considered. The isocyanate compound according to the present disclosure has electrophilicity. Therefore, when the isocyanate compound comes into contact with the block copolymer, the electrons of the epoxy group (crosslinked part) are peeled off, the bond is broken, and the epoxy group is ring-opened. The ring-opened epoxy groups react with each other, and the block copolymers are crosslinked (bonded) with each other. Alternatively, the ring-opened epoxy group of the block copolymer and the isocyanate group of the isocyanate compound react with each other, and the block copolymers are crosslinked (bonded) via the isocyanate compound. Therefore, the film strength of the surface lubricating layer is increased. Since the ring-opening reaction of the epoxy group is electron transfer between molecules, the isocyanate compound and the block copolymer (hydrophilic polymer molecule) need to approach each other until the electron orbitals of the molecules overlap. However, in the coating liquid, since the solvent is interposed between the isocyanate compound and the block copolymer, the isocyanate compound and the block copolymer are not in close contact with each other but exist apart from each other. Therefore, the isocyanate compound does not attract electrons of the epoxy group (crosslinked part), and ring-opening (crosslinking reaction) of the epoxy group hardly proceeds or does not proceed at all in the coating liquid. Since the ring-opening of the epoxy group of the block copolymer does not proceed, the coating liquid does not have high viscosity or gelation and can maintain the form of the solution. Therefore, the coating liquid can be uniformly applied onto the base material layer. On the other hand, when the coating liquid is applied onto the base material layer, and then the solvent is removed from the coating film of the coating liquid, the isocyanate compound comes into contact with the block copolymer to attract electrons (electron orbitals of molecules overlap), and ring-opening (crosslinking reaction) of the epoxy group is initiated and promoted. According to the method of the present disclosure, the coating liquid can be uniformly applied onto the base material layer, and the ring-opening (crosslinking reaction) of the epoxy group uniformly and densely proceeds on the surface of the base material layer, so that the film strength of the surface lubricating layer can be increased, and the block copolymer can be more firmly fixed to the surface of the base material layer. As a result, it is presumed that a surface lubricating layer (strong cover layer) having high film strength is formed, the strong cover layer can be favorably maintained even after sliding against a living body lumen such as a narrow blood vessel having higher flexibility, and a high lubricating property (surface lubricating property) can be retained for a longer period of time (that is, an excellent surface lubricating property can be retained, and sliding durability can be improved). Note that the above mechanism is an inference and does not limit the technical scope of the present disclosure.

Therefore, the medical device manufactured according to the method of the present disclosure has excellent durability (sliding durability). Since the medical device manufactured according to the method of the present disclosure also has excellent lubricating property and the durability of the lubricating property is also high, the medical device also has excellent surface lubrication retaining property.

### Method for Manufacturing Medical Device

The method for manufacturing a medical device according to the present disclosure includes: preparing a coating liquid containing a block copolymer having a structural unit (A) derived from a reactive monomer having an epoxy group and a structural unit (B) derived from a hydrophilic monomer, an isocyanate compound having two or more isocyanate groups, and a solvent ((I) preparation step); and applying the coating liquid onto the base material layer ((II) coating step). As described above, by using a solution containing a specific isocyanate compound together with the block copolymer as a coating liquid, a medical device having a surface lubricating layer (cover layer) that exhibits excellent durability can be obtained.

After the step (II), steps such as a step of performing drying and/or heat treatment ((III) drying/heat treatment step) and a step of washing ((IV) washing step) may be further performed as necessary. Among them, it is preferable to perform at least a (III) drying/heat treatment step after the step (II). If necessary, the (IV) washing step may be performed after the (III) drying/heat treatment step, but in the method of the present disclosure, since the isocyanate compound used in forming of a surface lubricating layer is covalently bonded to the block copolymer or becomes water-insoluble polyurea, it is not particularly necessary to perform the (IV) washing step, which is advantageous in mass production.

### (I) Preparation Step

In this step, a coating liquid containing a block copolymer, an isocyanate compound, and a solvent is prepared. Here, in this step, a coating liquid may be prepared by mixing a block copolymer, an isocyanate compound, and a solvent to prepare a coating liquid. Alternatively, a coating liquid may be prepared by mixing an isocyanate compound and a solvent to prepare a liquid containing the isocyanate compound, and then further mixing a block copolymer with the liquid containing the isocyanate compound to prepare a coating liquid. Alternatively, a coating liquid may be prepared by mixing a block copolymer and a solvent to prepare a liquid containing the block copolymer, and then further mixing an isocyanate compound with the liquid containing the block copolymer to prepare a coating liquid. Furthermore, a coating liquid containing a block copolymer, an isocyanate compound, and a solvent may be purchased, and the coating liquid may be used.

Hereinafter, preferred aspects of preparing a coating liquid by mixing a block copolymer, an isocyanate compound, and a solvent to prepare the coating liquid will be described in detail.

### Block Copolymer

In the present disclosure, the block copolymer forms a surface lubricating layer supported on at least a part of the base material layer. That is, in the medical device obtained by the method according to the present disclosure, the surface lubricating layer contains a block copolymer. The term “supporting” means a state in which the surface lubricating layer is fixed in a state of not being easily released from the surface of the base material layer, and includes not only a form in which the entire surface of the base material layer is completely covered with the surface lubricating layer, but also a form in which only a part of the surface of the base material layer is covered with the surface lubricating layer, that is, a form in which the surface lubricating layer is attached to only a part of the surface of the base material layer.

The block copolymer according to the present disclosure has the structural unit (A) derived from a reactive monomer having an epoxy group and the structural unit (B) derived from a hydrophilic monomer.

The reactive monomer having an epoxy group constituting the block copolymer has an epoxy group as a reactive group. The epoxy group is ring-opened to promote crosslinking (bonding) between the block copolymers, and the film strength of the surface lubricating layer is increased by introducing the structural unit (A) derived from such a reactive monomer into the block copolymer. When the base material layer is a resin material, the ring-opened epoxy group also causes crosslinking (bonding) between the block copolymer and the base material layer.

The reactive monomer constituting the block copolymer is not particularly limited as long as it has an epoxy group, and a known compound can be used. Among them, the reactive monomer having an epoxy group preferably includes at least one selected from the group consisting of glycidyl acrylate, glycidyl methacrylate (GMA), 3,4-epoxycyclohexyl methyl acrylate, 3,4-epoxycyclohexyl methyl methacrylate, β-methyl glycidyl methacrylate, and allyl glycidyl ether because crosslinking or polymerization of the block copolymer can be easily controlled. Among them, glycidyl (meth)acrylate is more preferable, and glycidyl methacrylate is particularly preferable in consideration of further promoting the crosslinking reaction, ease of manufacture, and the like.

The reactive monomer may be used singly or in combination of two or more types of the reactive monomer. That is, the reactive moiety derived from the reactive monomer may be a homopolymer type composed of one reactive monomer alone or a copolymer type composed of two or more reactive monomers. Note that, in the case of using two or more kinds, the form of the reactive moiety may be a block copolymer or a random copolymer.

That is, in a preferred embodiment of the present disclosure, the reactive monomer having an epoxy group includes at least one selected from the group consisting of glycidyl acrylate, glycidyl methacrylate, 3,4-epoxycyclohexyl methyl acrylate, 3,4-epoxycyclohexyl methyl methacrylate, β-methyl glycidyl methacrylate, and allyl glycidyl ether, or is at least one selected from the above group. In a more preferred embodiment of the present disclosure, the reactive monomer having an epoxy group is at least one of glycidyl acrylate and glycidyl methacrylate. In a particularly preferred embodiment of the present disclosure, the reactive monomer having an epoxy group is glycidyl methacrylate.

The hydrophilic monomer constituting the block copolymer has a swelling property upon contact with a body fluid or an aqueous solvent and therefore imparts a lubricating property (surface lubricating property) to the medical device. Therefore, by introducing the structural unit (B) derived from a hydrophilic monomer into the block copolymer, the lubricating property (surface lubricating property) of the medical device is improved, and friction when the medical device comes into contact with a lumen wall such as a blood vessel wall can be reduced.

The hydrophilic monomer constituting the block copolymer is not particularly limited as long as it has the above characteristics, and a known compound can be used. Examples of hydrophilic monomer include acrylamide and derivatives of acrylamide, vinylpyrrolidone, acrylic acid and methacrylic acid and derivatives of acrylic acid and methacrylic acid, polyethylene glycol acrylate and derivatives of polyethylene glycol acrylate, a monomer having a sugar or a phospholipid in a side chain and a water-soluble monomer such as maleic anhydride. More specifically, examples of the hydrophilic monomer include acrylic acid, methacrylic acid, N-methylacrylamide, N,N-dimethylacrylamide (DMAA), acrylamide, acryloylmorpholine, N, N-dimethylaminoethyl acrylate, N-vinylpyrrolidone, 2-methacryloyloxyethyl phosphorylcholine, 2-methacryloyloxyethyl-D-glycoside, 2-methacryloyloxyethyl-D-mannoside, vinyl methyl ether, 2-hydroxyethyl (meth)acrylate, 4-hydroxybutyl (meth)acrylate, 2-hydroxypropyl (meth)acrylate, 2-hydroxybutyl (meth)acrylate, 6-hydroxyhexyl (meth)acrylate, 1,4-cyclohexanedimethanol mono(meth)acrylate, 1-chloro-2-hydroxypropyl (meth)acrylate, diethylene glycol mono(meth)acrylate, 1,6-hexanediol mono(meth)acrylate, pentaerythritol tri(meth)acrylate, dipentaerythritol penta(meth)acrylate, neopentyl glycol mono(meth)acrylate, trimethylolpropane di(meth)acrylate, trimethylolethane di(meth)acrylate, 2-hydroxy-3-phenyloxypropyl (meth)acrylate, 4-hydroxycyclohexyl (meth)acrylate, 2-hydroxy-3-phenyloxy(meth)acrylate, 4-hydroxycyclohexyl (meth)acrylate, cyclohexanedimethanol mono(meth)acrylate, poly(ethylene glycol) methyl ether acrylate, and poly(ethylene glycol) methyl ether methacrylate. From the viewpoint of imparting an excellent lubricating property, ease of synthesis, and operability, the hydrophilic monomer preferably includes at least one selected from the group consisting of N,N-dimethylacrylamide, acrylamide, 2-hydroxyethyl methacrylate, and N-vinylpyrrolidone, and more preferably is at least one kind selected from the group consisting of N, N-dimethylacrylamide, acrylamide, and 2-hydroxyethyl methacrylate. Among them, from the viewpoint of excellent lubricating property, the hydrophilic monomer is particularly preferably N,N-dimethylacrylamide.

The hydrophilic monomer may be used singly or in combination of two or more types of the hydrophilic monomer. That is, the hydrophilic moiety derived from the hydrophilic monomer may be a homopolymer type composed of one hydrophilic monomer or a copolymer type composed of two or more hydrophilic monomers. Note that, in the case of using two or more kinds, the form of the hydrophilic moiety may be a block copolymer or a random copolymer.

That is, in a preferred embodiment of the present disclosure, the hydrophilic monomer includes at least one selected from the group consisting of N,N-dimethylacrylamide, acrylamide, 2-hydroxyethyl methacrylate, and N-vinylpyrrolidone, or is at least one selected from the above group. In a more preferred embodiment of the present disclosure, the hydrophilic monomer is at least one kind selected from the group consisting of N,N-dimethylacrylamide, acrylamide, and 2-hydroxyethyl methacrylate. In a particularly preferred embodiment of the present disclosure, the hydrophilic monomer is N,N-dimethylacrylamide.

The block copolymer has the structural unit (A) and the structural unit (B). Here, the ratio between the structural unit (A) and the structural unit (B) is not particularly limited as long as the above effect is obtained. In consideration of a good lubricating property, a lubrication retaining property, strength of the cover layer, bondability to the base material layer, and the like, the ratio of the structural unit (A) to the structural unit (B) (molar ratio of the structural unit (A): the structural unit (B)) is preferably 1:2 to 1:100, more preferably 1:2 to 1:50, still more preferably 1:5 to 1:50, and particularly preferably 1:10 to 1:30. Within such a range, the surface lubricating layer can sufficiently exhibit the lubricating property by the structural unit (B), and can exhibit sufficient cover layer strength, bondability to the base material layer (in the case of the resin material), and durability by the structural unit (A). The molar ratio of the structural unit (A): the structural unit (B) can be controlled by adjusting the charging ratio (molar ratio) of each monomer in the manufacture stage of the block copolymer. Therefore, the charging ratio (molar ratio) between the reactive monomer having an epoxy group and the hydrophilic monomer in the manufacture stage of the block copolymer is preferably 1:2 to 1:100, more preferably 1:2 to 1:50, still more preferably 1:5 to 1:50, and particularly preferably 1:10 to 1:30. Note that the molar ratio of the structural unit (A): the structural unit (B) can be confirmed, for example, by performing NMR measurement (¹H-NMR measurement, ¹³C-NMR measurement, etc.) on the copolymer.

The block copolymer according to the present disclosure essentially includes the structural unit (A) and the structural unit (B) but may have other structural units in addition to these structural units. When the block copolymer has other structural units, examples of the other structural units include adipic acid, glutaric acid, triethylene glycol, and tripropylene glycol. The monomers constituting other structural units may be used singly or may be used in combination of two or more of the monomers. That is, the other structural unit may be a homopolymer type composed of one kind of structural unit alone or a copolymer type composed of two or more kinds of structural units. When two or more monomers constituting the other structural unit are used, the segment constituted by the monomers may be in the form of a block copolymer, a random copolymer, or an alternating copolymer.

When the block copolymer according to the present disclosure has other structural units, the content of the other structural units is preferably more than 0 mol % and 5 mol % or less with respect to all the structural units constituting the block copolymer. That is, in the block copolymer according to the present disclosure, when the total of all structural units constituting the block copolymer is 100 mol %, the total of the contents of the structural unit (A) and the structural unit (B) is preferably 95 mol % or more (upper limit: less than 100 mol %). More preferably, the block copolymer according to the present disclosure is substantially composed of the structural unit (A) and the structural unit (B) (the content of other structural units=more than 0 mol % and less than 5 mol %). In this form, the block copolymer according to the present disclosure can achieve durability by the structural unit (A) and the lubricating property (surface lubricating property) by the structural unit (B) in a well-balanced manner. Preferably, the block copolymer according to the present disclosure does not include the other structural unit (content of other structural unit=0 mol %).

The composition of each structural unit (structural units (A) and (B) and other structural units) can be measured by a known method. For example, the composition (molar ratio) of the structural unit can be measured by measuring the integral ratio of the intensity of each signal in the 1H-NMR spectrum of the block copolymer solution.

In an embodiment of the present disclosure, the block copolymer according to the present disclosure is substantially composed of a structural unit (A) derived from at least one reactive monomer selected from the group consisting of glycidyl acrylate, glycidyl methacrylate, 3,4-epoxycyclohexyl methyl acrylate, 3,4-epoxycyclohexyl methyl methacrylate, β-methyl glycidyl methacrylate, and allyl glycidyl ether, and a structural unit (B) derived from at least one hydrophilic monomer selected from the group consisting of N, N-dimethylacrylamide, acrylamide, 2-hydroxyethyl methacrylate, and N-vinylpyrrolidone, or is composed only of the above.

In an embodiment of the present disclosure, the block copolymer according to the present disclosure is substantially composed of a structural unit (A) derived from a reactive monomer of at least one of glycidyl acrylate and glycidyl methacrylate, and a structural unit (B) derived from at least one hydrophilic monomer selected from the group consisting of N, N-dimethylacrylamide, acrylamide, and 2-hydroxyethyl methacrylate, or is composed only of the above.

In an embodiment of the present disclosure, the block copolymer according to the present disclosure is substantially composed of a structural unit (A) derived from glycidyl methacrylate (reactive monomer having an epoxy group) and a structural unit (B) derived from N,N-dimethylacrylamide (hydrophilic monomer), or is composed only of the above.

The weight average molecular weight of the block copolymer is preferably 10,000 or more and 10,000,000 or less from the viewpoint of solubility. The weight average molecular weight of the block copolymer is more preferably 100,000 or more and 5,000,000 or less from the viewpoint of ease of preparation of a coating liquid. In the present specification, as the “weight average molecular weight”, a value measured by gel permeation chromatography (Gel Permeation Chromatography, GPC) using polystyrene as a reference material is adopted.

The method for manufacturing the block copolymer is not particularly limited, and the block copolymer can be produced by applying a conventionally known polymerization method such as a living radical polymerization method, a polymerization method using a macro initiator, or a polycondensation method. Among them, a living radical polymerization method or a polymerization method using a macro initiator is preferably used because it is easy to control the molecular weight and molecular weight distribution of the structural unit (moiety) derived from a reactive monomer and the structural unit (moiety) derived from a hydrophilic monomer. The living radical polymerization method is not particularly limited, but for example, methods described in Japanese Patent Application Publication Nos. H11-263819 A, 2002-145971 A, 2006-316169 A, an atom transfer radical polymerization (ATRP) method, and the like can be applied in the same manner or appropriately modified. In a polymerization method using a macro initiator, for example, a macro initiator having a reactive moiety having a reactive functional group and a radical polymerizable group such as a peroxide group is produced, and then a monomer for forming a hydrophilic moiety is polymerized with the macro initiator, whereby a block copolymer having a hydrophilic moiety and a reactive moiety can be produced.

The block copolymer after polymerization is preferably purified by a general purification method such as a reprecipitation method, a dialysis method, an ultrafiltration method, or an extraction method.

### Isocyanate Compound Having Two or More Isocyanate Groups

In the present disclosure, the isocyanate compound has two or more isocyanate groups. The isocyanate compound has electrophilicity. Therefore, when the isocyanate compound and the block copolymer come into contact with each other, electrons of the epoxy group (crosslinked part) of the block copolymer are peeled off to induce ring-opening of the epoxy group. The epoxy group is ring-opened. The ring-opened epoxy groups react with each other, and the block copolymers are crosslinked (bonded) with each other. Alternatively, the ring-opened epoxy group of the block copolymer and the isocyanate group of the isocyanate compound react with each other, and the block copolymers are crosslinked (bonded) via the isocyanate compound. Therefore, the film strength of the surface lubricating layer is increased. When the base material layer is a resin material, the ring-opened epoxy group also causes crosslinking (bonding) between the block copolymer and the base material layer. Therefore, in the medical device obtained by the method according to the present disclosure, durability (sliding durability) can be further improved, and the shape can be favorably maintained even after sliding.

The promotion of crosslinking of the block copolymer as described above may also occur by the addition of an acid or a base to the coating liquid. However, in such a case, crosslinking or polymerization between the block copolymers rapidly proceeds in the coating liquid due to an acid or a base present in the coating liquid, so that the viscosity of the coating liquid becomes high (or gelled), and it may be difficult to uniformly apply the coating liquid. On the other hand, in the isocyanate compound, since the contact with the block copolymer is inhibited by the solvent in the coating liquid, crosslinking or polymerization of the block copolymer is less likely to proceed in the coating liquid. The isocyanate compound promotes the crosslinking reaction when the solvent is removed by heating and drying or the like and the isocyanate compound comes into contact with the block copolymer. Therefore, it is possible to suppress an increase in viscosity (gelation) of the coating liquid due to crosslinking of the block copolymer in the coating liquid as described above. Therefore, according to the method of the present disclosure, the coating liquid can be uniformly applied onto the base material layer. In addition, when an acid or a base is added to the coating liquid, it is necessary to perform an operation such as a washing step of removing the acid or the base remaining in the surface lubricating layer or a neutralization step of neutralizing the acid or the base remaining in the surface lubricating layer after forming the surface lubricating layer. On the other hand, in the method of the present disclosure, the compound used in forming of the surface lubricating layer is an isocyanate compound such as hexamethylene diisocyanate, toluene diisocyanate, or 4,4′-diphenylmethane diisocyanate. Therefore, since the above-described process is not separately required, the work efficiency can be enhanced.

In the present specification, the term “electrophilicity” refers to a property on an electron-receiving side, i.e., an electron-withdrawing side in a reaction for generating a chemical bond while electron exchange is performed between different chemical species.

The isocyanate compound according to the present disclosure may have two or more isocyanate groups, and is preferably, for example, an aliphatic isocyanate compound or aromatic isocyanate compound having 4 carbon atoms to 30 carbon atoms, more preferably an aliphatic isocyanate compound or aromatic isocyanate compound having 5 carbon atoms to 25 carbon atoms, still more preferably an aliphatic isocyanate compound or aromatic isocyanate compound having 8 carbon atoms to 22 carbon atoms, particularly preferably an aliphatic isocyanate compound or aromatic isocyanate compound having 10 carbon atoms to 20 carbon atoms, and most preferably an aliphatic isocyanate compound or aromatic isocyanate compound having 12 carbon atoms to 18 carbon atoms. When the number of carbon atoms is 4 to 30, sufficient solvent solubility is obtained, and thus coating properties are excellent. Among them, the aromatic isocyanate compound has higher electrophilicity so that crosslinking by the structural unit (A) of the block copolymer (particularly during thermal treatment) can be further promoted (durability can be further improved). In an embodiment, the isocyanate compound is an aliphatic or aromatic isocyanate compound having 6 carbon atoms to 20 carbon atoms. In an embodiment, the isocyanate compound is an aromatic isocyanate compound having 8 carbon atoms to 20 carbon atoms (preferably 10 carbon atoms to 18 carbon atoms). In an embodiment, the isocyanate compound is an aliphatic isocyanate compound having 4 to 20 (preferably 6 to 16) carbon atoms. In an embodiment, the isocyanate compound is an aromatic isocyanate compound having 10 carbon atoms to 20 carbon atoms. Note that the number of carbon atoms of the isocyanate compound also includes the carbon atoms of the two isocyanate groups of the isocyanate compound.

The isocyanate compound according to the present disclosure preferably has two or more and five or less isocyanate groups, more preferably two or more and four or less isocyanate groups, still more preferably two or more and three or less isocyanate groups, and particularly preferably two isocyanate groups.

Examples of the isocyanate compound according to the present disclosure include aliphatic diisocyanate compounds such as ethylene diisocyanate, hexamethylene diisocyanate, cyclohexylene diisocyanate, methylenebis(4,1-cyclohexylene)diisocyanate, 1,3-bis(isocyanatomethyl)cyclohexane, bis(isocyanatomethyl)benzene, and 5-isocyanato-1-(isocyanatomethyl)-1,3,3-trimethylcyclohexane (isophorone diisocyanate); and aromatic diisocyanate compounds such as xylene diisocyanate, toluene diisocyanate, diphenylmethane diisocyanate, naphthalene diisocyanate, 2,2′-diphenylmethane diisocyanate (2,2′-MDI), 2,4′-diphenylmethane diisocyanate (2,4′-MDI), 4,4′-diphenylmethane diisocyanate (4,4′-MDI), phenylene diisocyanate, tris(4-isocyanatophenyl) methane, and toluene triisocyanate. Note that, in the present specification, when the group to which the isocyanate group is directly bonded is aliphatic, it is referred to as an “aliphatic isocyanate compound”, and when the group to which the isocyanate group is directly bonded is an aromatic ring, it is referred to as an “aromatic diisocyanate”. For example, in bis(isocyanatomethyl)benzene, since an isocyanate group is bonded to a benzene ring via an aliphatic(methylene group), it is referred to as an “aliphatic isocyanate compound”.

The isocyanate compound preferably includes at least one selected from the group consisting of hexamethylene diisocyanate, toluene diisocyanate, and 4,4′-diphenylmethane diisocyanate. The isocyanate compound more preferably is at least one selected from the group consisting of hexamethylene diisocyanate, toluene diisocyanate, and 4,4′-diphenylmethane diisocyanate.

### Preparation of Coating Liquid

A coating liquid is prepared using the block copolymer, the isocyanate compound, and the solvent. Here, since the isocyanate compound is stable in the coating liquid, it is preferable from the viewpoint of safety and simplicity of operation. In the coating liquid, a solvent is interposed between the isocyanate compound and the block copolymer, and the isocyanate compound and the block copolymer are not in close contact with each other but exist separately from each other. On the other hand, the ring-opening (crosslinking reaction) of the epoxy group is started and progressed by approaching the block copolymer until the electron orbitals of the molecules of the block copolymer and the isocyanate compound overlap each other and exchanging electrons. For this reason, in the coating liquid, the ring-opening (crosslinking reaction) of the epoxy group hardly proceeds or does not proceed at all, and the viscosity of the coating liquid hardly changes or does not change at all. Therefore, workability is excellent.

The order of addition and the method of addition of the block copolymer, the isocyanate compound, and the solvent are not particularly limited. The above components may be added collectively or separately, stepwise or continuously. The mixing method is not particularly limited, and a known method can be used. Examples of the method for preparing the coating liquid include a method of sequentially adding an isocyanate compound and a block copolymer to a solvent, a method of sequentially adding a block copolymer and an isocyanate compound to a solvent, and a method of collectively adding an isocyanate compound and a block copolymer to a solvent.

Preferably, the isocyanate compound and the block copolymer are sequentially added into the solvent, or the block copolymer and the isocyanate compound are sequentially added into the solvent. If necessary, the above addition may be performed while stirring. Alternatively, the mixed solution may be stirred after the addition. In an embodiment, an isocyanate compound is added and mixed in a solvent, and then a block copolymer is further added and mixed to prepare a coating liquid.

The solvent used for preparing the coating liquid is not particularly limited as long as it can dissolve the block copolymer and the isocyanate compound (and other components, if used), and is appropriately selected according to the type of the block copolymer and the isocyanate compound (and other components, if used). Alcohol-based solvents such as methanol, ethanol, isopropyl alcohol and butanol from the viewpoint of high solubility; organic solvents such as dichloromethane, chloroform, carbon tetrachloride, tetrahydrofuran (THF), dimethyl sulfoxide, N,N-dimethylformamide (DMF), dioxane, and benzene are preferably used. In an embodiment, the solvent preferably contains at least one selected from the group consisting of tetrahydrofuran, N,N-dimethylformamide, dimethyl sulfoxide, and acetone. These may be used singly or in mixture of two or more types of the solvent (in a form of a mixed solvent).

The concentration of the block copolymer in the coating liquid is not particularly limited. From the viewpoint of further improving coatability, lubricating property and durability of the surface lubricating layer, the concentration of the block copolymer in the coating liquid is preferably 0.5 mass % or more and 20 mass % or less, more preferably 1 mass % or more and 15 mass % or less, still more preferably 2 mass % or more and 12 mass % or less, and particularly preferably 3 mass % or more and 10 mass % or less. The concentration is particularly preferably 3 mass % or more and 8 mass % or less. When the concentration of the block copolymer is within the above range, the lubricating property and durability of the resulting surface lubricating layer can be sufficiently exhibited. In addition, a uniform surface lubricating layer having a desired thickness can be easily obtained by one coating, and the viscosity of the solution falls within an appropriate range, which is preferable in terms of operability (ease of coating, for example) and production efficiency. However, even if it is out of the above range, it can be sufficiently used as long as it does not affect the operation and effect of the present disclosure.

The concentration of the isocyanate compound in the coating liquid is also not particularly limited. From the viewpoint of the lubricating property (sliding property) of the surface lubricating layer, it is preferable that the addition amount of the isocyanate compound is small. On the other hand, from the viewpoint of durability (sliding durability) of the surface lubricating layer, the isocyanate compound is preferably added to some extent. From the above viewpoint, the concentration of the isocyanate compound in the coating liquid is preferably 0.001 mass % or more and 12 mass % or less, more preferably 0.005 mass % or more and 5.0 mass % or less, still more preferably 0.01 mass % or more and less than 3.0 mass %, particularly preferably 0.01 mass % or more and 1.0 mass % or less, and most preferably 0.05 mass % or more and 0.8 mass % or less. When the concentration of the isocyanate compound is within the above range, the crosslinking of the block copolymer is not allowed to proceed excessively while sufficiently securing the lubricating property (sliding property) of the surface lubricating layer (crosslinking can be moderately promoted). Therefore, the lubricating property and durability of the resulting surface lubricating layer can be sufficiently exhibited. However, even if it is out of the above range, it can be sufficiently used as long as it does not affect the operation and effect of the present disclosure.

The mixing ratio of the block copolymer and the isocyanate compound in the coating liquid is such that the content of the isocyanate compound in the coating liquid is preferably equal to or less than the content of the block copolymer in the coating liquid from the viewpoint of further improvement in durability (sliding durability), coatability, the lubricating property of the surface lubricating layer, and the like, and is more preferably less than the content of the block copolymer in the coating liquid from the viewpoint of further improvement in durability (sliding durability). More specifically, the mixing ratio of the block copolymer and the isocyanate compound (block copolymer: isocyanate compound (mass ratio)) in the coating liquid can be, for example, 100:0.01 or more and 100:30 or less, preferably 100:0.02 or more and 100:25 or less, more preferably 100:0.1 or more and 100:20 or less, still more preferably 100:0.2 or more and 100:10 or less, still more preferably 100:0.3 or more and 100:9 or less, particularly preferably 100:0.5 or more and 100:8 or less, and most preferably 100:1 or more and 100:5 or less. When the mass ratio of the block copolymer and the isocyanate compound is within the above range, the durability and lubricating property of the resulting surface lubricating layer can be sufficiently exhibited.

### (II) Coating Step

In this step, the coating liquid prepared in the (I) preparation step is applied onto the base material layer to form a coating film (coating layer) on the base material layer.

The base material layer may be made of any material, and examples of the base material layer can include a metal material, a polymer material (resin material), and ceramics.

Among the materials constituting the base material layer, the metal material is not particularly limited, and metal materials generally used for medical devices such as a catheter, a guide wire, and an indwelling needle are used. Specific examples of the materials constituting the base material layer can include various stainless steels (SUS) such as SUS304, SUS314, SUS316, SUS316L, SUS420J2, and SUS630, gold, platinum, silver, copper, nickel, cobalt, titanium, iron, aluminum, tin, or various alloys such as a nickel-titanium alloy, a nickel-cobalt alloy, a cobalt-chromium alloy, and a zinc-tungsten alloy. These may be used singly or in combination of two or more types of the materials. As the metal material, an optimal metal material may be appropriately selected as a base material layer of a catheter, a guide wire, an indwelling needle, or the like which is used.

Among the materials constituting the base material layer, the polymer material (resin material or elastomer material) is not particularly limited, and a polymer material generally used for medical devices such as a catheter, an introducer, a guide wire, and an indwelling needle is used. Specific examples of the materials of the base material layer can include a polyamide resin (nylon), polyolefin resins such as a polyethylene resin and a polypropylene resin, a modified polyolefin resin, a cyclic polyolefin resin, an epoxy resin, a polyurethane resin, a diallyl phthalate resin (allyl resin), a polycarbonate resin, a fluororesin, an amino resin (a urea resin, a melamine resin, a benzoguanamine resin), a polyethylene terephthalate resin and polyester resins such as a polybutylene terephthalate resin, a styrol resin, an acrylic resin, a polyacetal resin, a vinyl acetate resin, a phenol resin, a vinyl chloride resin, a silicone resin (silicon resin), a polyether resin and a polyimide resin. Note that the resin also includes an elastomer. Therefore, thermoplastic elastomers such as a polyurethane elastomer as a polyurethane resin, a polyester elastomer as a polyester resin, and a polyamide elastomer (nylon elastomer) as a polyamide resin (nylon) can also be used as the material of the base material layer.

These polymer materials may be used singly or may be used as a mixture of two or more of the polymer materials or as a copolymer of two or more monomers constituting any of the resin or elastomer. Among them, the polymer material is preferably a polyamide resin, a polyolefin resin, a polyester resin, or a polyurethane resin, more preferably a polyethylene resin, a polyester resin, a polyester elastomer, a polyurethane resin, a polyethylene terephthalate resin, a polyamide resin, or a polyamide elastomer, and still more preferably a polyethylene resin, a polyester resin, a polyester elastomer, a polyamide resin or a polyamide elastomer. A carboxy group or an amino group as an end group included in the polyamide resin or the polyamide elastomer may undergo a crosslinking reaction with an epoxy group in the block copolymer. These polymer materials (particularly, polyamide resin and polyamide elastomer) are relatively soft, and it can be said that the block copolymer constituting the surface lubricating layer easily penetrates into these polymer materials. Therefore, the bondability between the polymer material (particularly, the polyamide resin and the polyamide elastomer) and the block copolymer is enhanced, and a surface lubricating layer having more excellent durability can be formed. As the polymer material, a polymer material optimal for a base material layer of a catheter, a guide wire, an indwelling needle, or the like that is used may be appropriately selected.

According to the manufacture method of the present disclosure, since a surface lubricating layer excellent in durability can be formed by a heat treatment at a low temperature, for example, even in the case of a base material layer having low solubility, a surface lubricating layer excellent in durability can be formed. Examples of such a base material layer include a metal, a polyamide resin, a polyolefin resin, a polyester resin, and a polyurethane resin, and among these, a metal, a polyethylene resin, a polyester elastomer, a polyamide elastomer, and the like are preferable. Therefore, according to an embodiment, the base material layer is one or more selected from the group consisting of a metal, a polyethylene resin, a polyester elastomer, and a polyamide elastomer.

The shape of the base material layer is not particularly limited and is appropriately selected according to a use mode such as a sheet shape, a linear shape (wire), a rod shape, or a tubular shape.

The method for applying the coating liquid to the surface of the base material layer is not particularly limited, and conventionally known methods such as a coating/printing method, an immersion method (dipping method, dip coating method), a spraying method, a spin coating method, a mixed solution impregnation sponge coating method, a bar coating method, a die coating method, a reverse coating method, a comma coating method, a gravure coating method, and a doctor knife method can be applied. Among them, an immersion method (dipping method, dip coating method) is preferably used.

When the surface lubricating layer is formed only on a part of the base material layer, the surface lubricating layer can be formed on a desired surface portion of the base material layer by immersing only a part of the base material layer in a coating liquid and coating the part of the base material layer with the coating liquid.

The coating amount of the coating liquid is preferably such an amount that the thickness (dry film thickness) of the resulting coating (surface lubricating layer) is 0.1 μm to 10 μm, more preferably such an amount that the thickness is 0.5 μm to 5 μm, and still more preferably such an amount that the thickness is 1 μm to 3 μm. When the coating amount is such that the thickness of the coating (surface lubricating layer) is 0.1 μm or more, the durability of the obtained coating (surface lubricating layer) can be sufficiently achieved. When the coating amount is such that the thickness of the coating (surface lubricating layer) is 10 μm or less, the surface of the coating (surface lubricating layer) is less likely to be sticky, and handling at the time of manufacturing becomes easier.

### (III) Drying/Heat Treatment Step

In the method for manufacturing a medical device according to the present disclosure, if necessary, a coating film (coating layer) may be formed by coating a coating liquid onto the base material layer in the (II) coating step, and then a drying step and/or a heat treatment step may be performed. For the purpose of removing the solvent and forming a strong surface lubricating layer, it is preferable to perform a drying step and/or a heat treatment step, and it is more preferable to perform the drying step and the heat treatment step.

Here, although the “drying treatment” and the “heat treatment” are not strictly distinguished from each other, for convenience of description, the “drying treatment” refers to a “drying treatment” of holding the base material layer to which the coating liquid is applied at a temperature (20° C. to 30° C.) or lower around room temperature, and the “heat treatment” refers to a “heat treatment” of holding the base material layer at a temperature exceeding the temperature (20° C. to 30° C.) around room temperature.

Here, in the manufacture method of the present disclosure, a drying treatment is preferably performed before the heat treatment. The drying treatment before the heat treatment is performed in order to volatilize the solvent to some extent for the purpose of improving the handleability of the coating film formed on the base material layer before the heat treatment as the subsequent step is performed.

The conditions during the drying treatment before the heat treatment are not particularly limited but are preferably conditions under which the solvent volatilizes to such an extent that the coating film formed on the base material layer can maintain a certain shape. For example, the temperature of the drying treatment before the heat treatment is preferably 10° C. or higher and 30° C. or lower. The time for the drying treatment before the heat treatment is preferably 0.5 hours or more and 3 hours or less. That is, it is more preferable to maintain the coating film formed by applying the block copolymer solution onto the base material layer at 10° C. or higher and 30° C. or lower for 0.5 hours or more and 3 hours or less. Note that the temperature may be changed during the drying treatment.

The temperature of the heat treatment is preferably lower than 100° C., more preferably higher than 30° C. and lower than 100° C., still more preferably 35° C. or higher and 95° C. or lower, particularly preferably 40° C. or higher and 90° C. or lower, and most preferably 50° C. or higher and 85° C. or lower. In an embodiment, the temperature of the heat treatment is 80° C. or higher and lower than 100° C. When the temperature is maintained at such a temperature (thermal energy is added), electron transfer more actively occurs due to expansion and contraction of molecules by thermal energy, ring-opening (crosslinking reaction) of the epoxy group is further promoted, a surface lubricating layer having higher durability can be formed, and the surface lubricating layer can exhibit excellent lubricating property. Damage to the hydrophilic group in the structural unit (B) of the base material layer (reduction in hydrophilicity of the structural unit (B) of the block copolymer) can be suppressed, so that the surface lubricating layer can exhibit better lubricating property. In particular, by setting the heating temperature to lower than 100° C., when the base material layer is made of the polymer material (resin material or elastomer material), the base material layer can be suppressed from being deformed or plasticized by heat. Therefore, according to the present disclosure, the selectivity of the material is further widened, and medical devices for various applications can be manufactured. Since it is possible to form the surface lubricating layer having excellent durability at a low temperature, it is preferable from the viewpoint of energy cost at the time of manufacturing a medical device. Note that the temperature may be changed during the heat treatment.

When the base material layer is a metal, the heat treatment time is preferably 10 hours or less, more preferably 9 hours or less, still more preferably 8 hours or less, particularly preferably 6 hours or less, and most preferably 5 hours or less. When the base material layer is a metal, the time for the heat treatment may be 0.5 hours or more, may be 1 hour or more, and is preferably 3 hours or more. When the base material layer is a resin, the heat treatment time is preferably 10 hours or less, more preferably 8 hours or less, still more preferably 6 hours or less, particularly preferably 5 hours or less, and most preferably 2 hours or less. When the base material layer is a resin, the heat treatment time may be 0.5 hours or more and may be 1 hour or more. Crosslinking or polymerization in the block copolymer is effectively promoted by setting such a time, and a strong cover layer (surface lubricating layer) is formed. Accordingly, it is possible to retain high lubricating property (surface lubricating property) for a longer period of time. The above time range prevents excessive progress of crosslinking or polymerization. Therefore, it is possible to prevent reduction in the swelling property due to excessive curing of the surface lubricating layer, thereby, it is possible to retain good lubricating property (surface lubricating property). By setting such a time, a decrease in hydrophilicity of the structural unit (B) of the block copolymer can be prevented or suppressed, and lubricating property can be more easily controlled. In particular, when the base material layer is made of the polymer material (resin material or elastomer material), the base material layer can be suppressed from being deformed or plasticized by heat as long as the heat treatment time is as short as 5 hours or less.

That is, as a preferred aspect of the present disclosure, a coating liquid is applied onto a base material layer to form a coating film, and then the coating film is heat-treated at a temperature of higher than 30° C. and lower than 100° C. for 10 hours or less (that is, the coating film is maintained). As a more preferred aspect, a coating liquid is applied onto a base material layer to form a coating film, and then the coating film is heat-treated at a temperature of 80° C. or higher and lower than 100° C. for 1 hour or more and 10 hours or less (that is, the coating film is maintained). Crosslinking or polymerization of the block copolymer is effectively promoted by maintaining the coating film under such conditions (time and temperature), and a strong cover layer (surface lubricating layer) is formed. Accordingly, it is possible to retain high lubricating property (surface lubricating property) for a longer period of time. It is possible to suppress excessive progress of the crosslinking or polymerization by maintaining the coating film under such conditions (time and temperature). Therefore, it is possible to prevent reduction in the swelling property due to excessive curing of the surface lubricating layer, thereby, it is possible to retain good lubricating property (surface lubricating property).

The conditions during the drying treatment after the heat treatment are not particularly limited as long as the conditions allow the surface lubricating layer including the block copolymer on the base material layer to be cooled to room temperature.

By performing the drying/heat treatment step under the conditions (temperature, time, etc.) as described above, a strong surface lubricating layer (cover layer) can be supported on the surface of the base material layer. Depending on the type of the base material layer, a crosslinking reaction via the epoxy group in the block copolymer in the surface lubricating layer occurs, and a high-strength surface lubricating layer that is not easily peeled off from the base material layer can be formed. Therefore, peeling of the surface lubricating layer from the base material layer can be effectively suppressed and prevented by the drying/heat treatment step.

The pressure condition during drying is not limited at all, and the drying may be performed under normal pressure (atmospheric pressure) or may be performed under pressure or reduced pressure.

As the drying or heating means (device), for example, an oven, a vacuum dryer, or the like can be used, but in the case of natural drying, the drying means (device) is particularly unnecessary.

### (IV) Washing Step

In the method for manufacturing a medical device according to the present disclosure, if necessary, a coating film (coating layer) may be formed by applying a coating liquid onto the base material layer in the (II) coating step, the (III) drying/heat treatment step may be performed, and then the surface lubricating layer provided on the base material layer may be washed ((IV) washing step). The washing step is performed for the purpose of efficiently removing impurities and unreacted substances contained in the surface lubricating layer and imparting more excellent lubricating property (low friction property) to the surface lubricating layer.

The washing method is not particularly limited, but a method of immersing the surface lubricating layer by the block copolymer in a washing solvent, a method of pouring the washing solvent, or a combination of the two methods may be used. The washing solvent used at this time is not particularly limited as long as it does not dissolve the surface lubricating layer by the block copolymer and can efficiently remove impurities and unreacted substances, but water (for example, ion-exchanged water, distilled water, reverse osmosis membrane water, filtered water, sterilized water, or purified water) or warm water is preferably used. The temperature of the washing water is not particularly limited, but is preferably 20° C. to 100° C., and more preferably 25 to 80° C. The washing time (time for bringing the washing solvent into contact with the coating) is not particularly limited, but is preferably 30 seconds to 60 minutes, and more preferably 1 minute to 30 minutes.

After the washing step, a drying step may be further performed. The drying method and the drying conditions (temperature, time, etc.) are not particularly limited, and conventionally known methods can be used.

A medical device manufactured by the method according to the present disclosure has a structure in which a solution (coating liquid) containing a block copolymer for forming a surface lubricating layer and an isocyanate compound is applied onto a base material layer, so that the surface lubricating layer is supported on the base material layer. Whether or not the isocyanate compound is contained in the surface lubricating layer can be confirmed by the following method. That is, it can be confirmed by extracting the surface lubricating layer using an organic solvent such as deuterated chloroform, analyzing the obtained extraction solution by NMR (¹H-NMR measurement, ¹³C-NMR measurement, or the like), LC-MS (liquid chromatography mass spectrometry), or GC (gas chromatography method), and detecting a peak derived from an isocyanate group of an isocyanate compound.

### Medical Device

Through the (I) preparation step and the (II) coating step described above, and the (III) drying/heat treatment step and/or the (IV) washing step performed as necessary, a medical device having a surface lubricating layer (cover layer) exhibiting excellent durability can be manufactured.

According to the method of the present disclosure, after forming the coating film including the block copolymer and the isocyanate compound on the surface of the base material layer, the epoxy group is crosslinked to form a strong surface lubricating layer that is not easily peeled off from the base material layer. At this time, since the epoxy group of the block copolymer is crosslinked with the isocyanate group of the isocyanate compound, a crosslinking reaction product of the epoxy group of the block copolymer and the isocyanate group of the isocyanate compound having two or more isocyanate groups is included. In the medical device obtained by the method according to the present disclosure, since the surface lubricating layer formed of the crosslinking reaction product of the block copolymer and the isocyanate compound is formed on the surface, excellent lubricating property and durability (lubrication retaining property) can be exhibited.

A medical device manufactured by the method according to the present disclosure has a structure in which a coating liquid including a block copolymer for forming a surface lubricating layer and an isocyanate compound is applied onto a base material layer, so that the surface lubricating layer is supported on the base material layer. In the coating liquid, the isocyanate compound and the block copolymer are dissolved in a solvent (the isocyanate compound and the block copolymer are uniformly mixed in the coating liquid). Therefore, the block copolymer and the isocyanate compound are interpenetrated (crosslinked) with each other in the surface lubricating layer.

Therefore, according to another aspect of the present disclosure, there is provided a medical device including: a base material layer; and a surface lubricating layer supported on at least a part of the base material layer, wherein the surface lubricating layer comprises a crosslinking reaction product of an epoxy group of a block copolymer having a structural unit (A) derived from a reactive monomer having an epoxy group and a structural unit (B) derived from a hydrophilic monomer and an isocyanate group of an isocyanate compound having two or more isocyanate groups. In this aspect, the terms such as the block copolymer and the isocyanate compound are the same as those described in Method for manufacturing medical device above, and thus the description of those terms is omitted here. As described above, the block copolymer and the isocyanate compound in the coating liquid are preferably contained at a mass ratio of 100:0.5 or more and 100:8 or less. Therefore, the mass ratio of the block copolymer and the isocyanate compound present in the surface lubricating layer is preferably 100:0.5 or more and 100:8 or less. At this time, the preferred abundance ratio (mass ratio) of the block copolymer and the isocyanate compound present in the surface lubricating layer is the same as the preferred mass ratio of the block copolymer and the isocyanate compound in the coating liquid.

Hereinafter, preferred embodiments of a medical device manufactured by the method according to the present disclosure will be described with reference to the accompanying drawings. Dimensional ratios in the drawings are exaggerated for convenience of description and may be different from actual ratios. In a case in which the embodiments of the present disclosure are described with reference to the drawings, the same elements are denoted by the same reference numerals in the description of the drawings, and redundant description will be omitted.

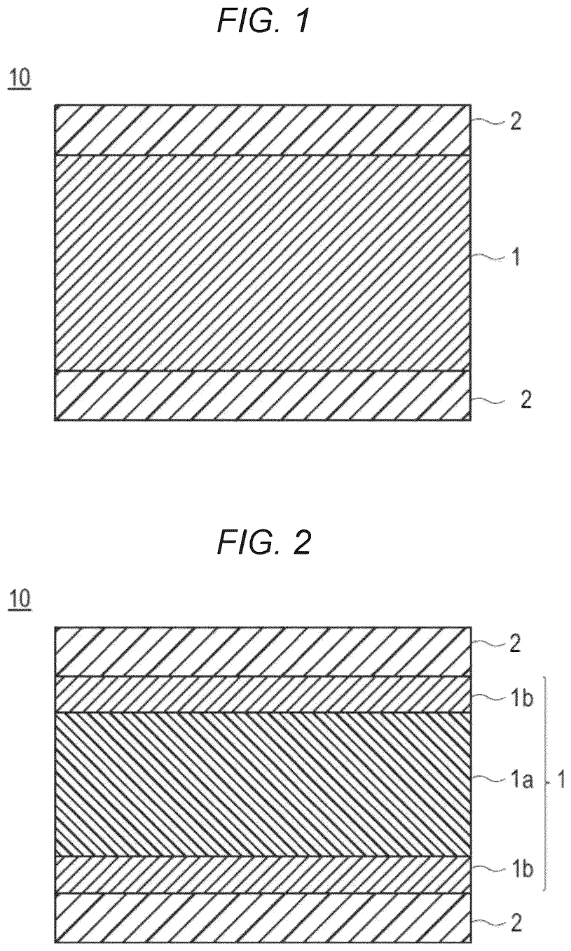

FIG. 1 is a partial cross-sectional view schematically illustrating a lamination structure of a surface of a representative embodiment of a medical device (in the present specification, it is also abbreviated as “medical device”) manufactured by a method according to the present disclosure. FIG. 2 is a partial cross-sectional view schematically illustrating a different structure example of the laminated structure on the surface as an application example of the present embodiment. Note that reference numerals in FIG. 1 and FIG. 2 indicate the following. Reference numeral 1 indicates a base material layer; reference numeral 1a indicates a base material layer core portion; reference numeral 1b indicates a base material surface layer; reference numeral 2 indicates a surface lubricating layer; and reference numeral 10 indicates a medical device manufactured by the method according to the present disclosure, respectively.

As illustrated in FIGS. 1 and 2, a medical device 10 of the present embodiment includes a base material layer 1 and a surface lubricating layer 2 provided on at least a part of the base material layer 1 and including a block copolymer (in the drawing, an example in which the film is provided on the entire surface (whole surface) of the base material layer 1 in the drawing is shown). In FIGS. 1 and 2, the surface lubricating layer 2 is formed on both surfaces of the base material layer 1, but the present disclosure is not limited to the above embodiment, and may be any form such as a form in which it is formed on one surface of the base material layer 1; and a form in which it is formed on a part of one surface or both surfaces of the base material layer 1.

Hereinafter, the medical device will be described in detail for each component.

### Base Material Layer (Base Material)

The base material layer used in the present embodiment may be composed of any material, and the material is not particularly limited. Specific examples of the material constituting the base material layer 1 include a metal material, a polymer material, and ceramics. Note that specific examples of the material constituting the base material layer 1 are as described in the (II) coating step.

Here, in the base material layer 1, the entire base material layer 1 may be made of any of the above materials. The base material layer 1 may be a multilayer structure formed by laminating different materials in multiple layers, a structure in which members formed of different materials are connected for each part of a medical device, or the like. Alternatively, as illustrated in FIG. 2, the base material layer core portion 1a may have a structure in which the base material surface layer 1b is formed by covering the surface of the base material layer core portion 1a formed of any of the above materials with any other of the above materials by an appropriate method. Examples of the latter case include: the layer in which the surface of the base material layer core portion 1a formed of a resin material or the like is covered with a metal material by an appropriate method (conventionally known methods such as plating, metal vapor deposition, and sputtering) to form the base material surface layer 1b; and the layer in which the surface of a base material layer core portion 1a formed of a hard reinforcing material such as a metal material or a ceramic material is covered with a polymer material that is softer than the reinforcing material such as a metal material by an appropriate method (conventionally known methods such as immersion (dipping), spraying, and coating/printing), or the reinforcing material that forms the base material layer core portion 1a and the polymer material are combined to form a base material surface layer 1b. Further, the base material layer core portion 1a may have a multilayer structure formed by laminating different materials in multiple layers, a structure in which members formed of different materials are connected for each part of the medical device, or the like. Another middle layer may be further formed between the base material layer core portion 1a and the base material surface layer 1b. The base material surface layer 1b may also have a multilayer structure formed by laminating different materials in multiple layers, a structure in which members formed of different materials are connected for each part of the medical device, or the like.

### Surface Lubricating Layer (Cover Layer)

The surface lubricating layer is supported on at least a part of the base material layer 1. Here, the reason why the surface lubricating layer 2 is supported on at least a part of the surface of the base material layer 1 is that, in a medical device such as a catheter, a guide wire, or an indwelling needle to be used, it is not always necessary that all surfaces (the entire surface) of these medical devices have the lubricating property in a wet state, and it is sufficient that the surface lubricating layer is supported only on a surface portion (which may be a part or all) where the surface is required to have the lubricating property in a wet state. Therefore, as described above, the surface lubricating layer includes: a form formed so as to cover the entire both surfaces of the base material layer as illustrated in FIGS. 1 and 2; a form formed so as to cover only entire one surface of base material layer; a form formed so as to cover a part of both surfaces of the base material layer in the same or different form; a form formed so as to cover a part of one surface of base material layer, and the like.

### Application of Medical Device

The medical device manufactured by the method of the present disclosure is a device used in contact with a body fluid, blood, or the like, and has a surface having the lubricating property in an aqueous liquid such as a body fluid or saline (or saline solution) and can improve operability and reduce damage to mucosal tissue. Specific examples of the medical devices can include a catheter, a guide wire, and an indwelling needle used in a blood vessel, and the following medical devices are also shown.
(a) Catheters inserted into or indwelled in a digestive organ orally or nasally, such as a gastric tube catheter, a feeding catheter, or a feeding tube.
(b) Catheters inserted or indwelled into or in the airway or trachea orally or nasally, such as an oxygen catheter, an oxygen cannula, a tube or cuff of an endotracheal tube, a tube or cuff of a tracheostomy tube, and an intratracheal aspiration catheter.
(c) Catheters inserted into or indwelled in the urethra or ureter, such as a urethral catheter, a urinary catheter, and catheter or balloon of a urethral balloon catheter.
(d) Catheters inserted into or indwelled in various body cavities, organs, and tissues such as suction catheters, drainage catheters, and rectal catheters.
(e) Catheters inserted into or indwelled in a blood vessel, such as an indwelling needle, an IVH catheter, a thermodilution catheter, an angiographic catheter, a vasodilator catheter, a dilator, or an introducer, or a guide wire, a stylet, or the like for these catheters.
(f) Artificial trachea, artificial bronchus, etc.
(g) Medical devices for extracorporeal circulation treatment (an artificial lung, an artificial heart, an artificial kidney, etc.) and circuits (e.g., blood flow circuits) of the medical devices.

### EXAMPLES

The effects of the present disclosure will be described with reference to the following Examples and Comparative Examples. However, the technical scope of the present disclosure is not limited only to the following Examples. Note that, in the following Examples, unless otherwise specified, an operation was performed at room temperature (25° C.). Unless otherwise specified, “%” and “parts” mean “mass %” and “parts by mass”, respectively.

### Synthesis Example 1

The following reaction was allowed to proceed, and a block copolymer (1) was manufactured.

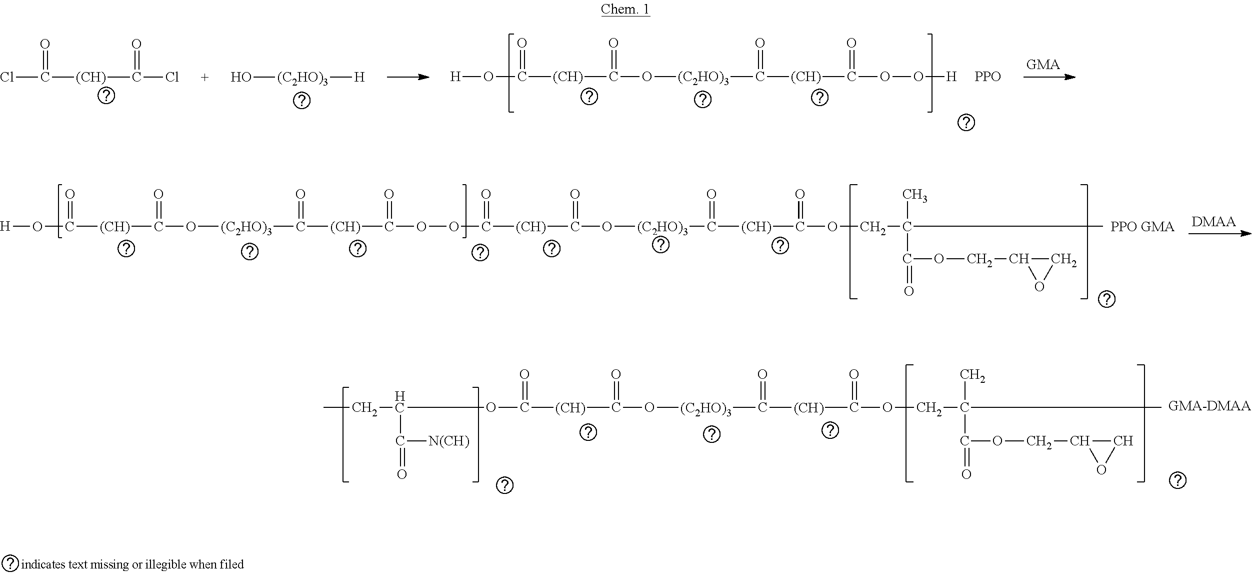

After 29.7 g of triethylene glycol was added dropwise to 72.3 g of adipic acid dichloride at 50° C., the mixture was reacted at 50° C. for 3 hours, and then hydrochloric acid was removed under reduced pressure to obtain an oligoester. Next, 4.5 g of methyl ethyl ketone was added to 22.5 g of the obtained oligoester, and the mixture was added dropwise to a solution containing 5 g of sodium hydroxide, 6.93 g of 31% hydrogen peroxide, 0.44 g of dioctyl phosphate as a surfactant, and 120 g of water and reacted at −5° C. for 20 minutes. The resulting product was repeatedly washed with water and methanol, and then, dried to obtain a poly peroxide (PPO) having a plurality of peroxide groups in a molecule.

Next, 0.5 g of this PPO and 9.5 g of glycidyl methacrylate (GMA) were polymerized in 30 g of benzene as a solvent while being stirred at 80° C. for 2 hours under reduced pressure. The reaction product obtained after the polymerization was reprecipitated with diethyl ether to obtain polyglycidyl methacrylate having a plurality of peroxide groups in a molecule (PPO-GMA).

Subsequently, 1.0 g of the obtained PPO-GMA (corresponding to 7 mmol of GMA) was charged into 9.0 g of N, N-dimethylacrylamide (DMAA) and 90 g of dimethyl sulfoxide as a solvent, and the mixture was reacted at 80° C. for 18 hours. The reaction product obtained after the reaction was reprecipitated with hexane and recovered to obtain a block copolymer (1) (structural unit (A): structural unit (B)=GMA:DMAA=1:14 (molar ratio)) having an epoxy group in the molecule and exhibiting lubricating property in a wet state. The block copolymer (1) thus obtained was analyzed by ¹H-NMR and ATR-IR, and it was confirmed that an epoxy group was present in the molecule. The weight-average molecular weight (Mw) of the block copolymer (1) measured by gel permeation chromatography (GPC, polystyrene equivalent) was about 1.5 million.

### Example 1

4,4′-Diphenylmethane diisocyanate (manufactured by KANTO CHEMICAL CO., INC., product name: methylenediphenyl 4,4′-diisocyanate) was added to N,N-dimethylformamide (DMF) so that the final concentration in the coating liquid was 0.1 mass %, and the mixture was slightly shaken and stirred to be dissolved, and it was visually confirmed that there was no solid undissolved, thereby obtaining a solution (1). Thereafter, the block copolymer (1) synthesized in Synthesis Example 1 was added to the solution (1) so that the final concentration in the coating liquid was 4.5 mass %, and the mixture was stirred for 1 hour using a stirrer. It was visually confirmed that there was no solid undissolved, thereby preparing a coating liquid (1).

A rod (outer diameter: 3.00 mm) made of SUS (manufactured by MISUMI Corporation, SUS304 Polishing Rod RGOS3-200) as a base material was immersed in the coating liquid (1), and the rod was dip-coated (pulling speed: 8 mm/sec). The rod to which the coating liquid (1) was attached was dried at room temperature (25° C.) for 1 hour to form a coating film (1) on the surface of the rod (rod (1)). Further, the rod (1) was stored in an oven at 80° C. for 5 hours, and the coating film (1) was heat-treated (rod (1′)). The rod (1′) was dried at room temperature (25° C.) for 1 hour to produce a sample (cover rod) (1) having a cover layer (surface lubricating layer) (dry film thickness: 1 μm) including a crosslinked copolymer derived from the block copolymer (1) on the surface.

### Example 2

A sample (cover tube) (2) was produced by the same method as in Example 1 except that in Example 1, the base material was changed from SUS to a nylon elastomer (manufactured by Evonik Industries, VESTAMID E47-S1) tube (outer diameter: 2.80 mm).

### Example 3

A sample (cover tube) (3) was produced by the same method as in Example 1 except that in Example 1, the base material was changed from SUS to a polyester elastomer (manufactured by TOYOBO Co., Ltd., PELPRENE E-450B) tube (outer diameter: 2.46 mm).

### Example 4

A sample (cover tube) (4) was produced by the same method as in Example 1 except that in Example 1, the base material was changed from SUS to a polyethylene (manufactured by AS ONE Corporation, Polyethylene tube hose 6-608-02) tube (outer diameter: 4.00 mm).

### Comparative Example 1

The block copolymer (1) synthesized in Synthesis Example 1 was dissolved in N, N-dimethylformamide (DMF) so that the final concentration in the coating liquid was 4.5 mass %, and the mixture was stirred for 1 hour using a stirrer. It was visually confirmed that there was no solid undissolved, thereby preparing a comparative coating liquid (1).

A rod (outer diameter: 3.00 mm) made of SUS (manufactured by MISUMI Corporation, SUS304 Polishing Rod RGOS3-200) as a base material was immersed in the comparative coating liquid (1), and the rod was dip-coated (pulling speed: 8 mm/sec). The rod to which the comparative coating liquid (1) was attached was dried at room temperature (25° C.) for 1 hour to form a comparative coating film (1) on the surface of the rod (comparative rod (1)). Further, the comparative rod (1) was stored in an oven at 80° C. for 5 hours, and the comparative coating film (1) was heat-treated (comparative rod (1′)). The comparative rod (1′) was dried at room temperature (25° C.) to produce a comparative sample (comparative cover rod) (1) having a cover layer (surface lubricating layer) (dry film thickness: 1 μm) including a crosslinked copolymer derived from the block copolymer (1) on the surface.

### Comparative Example 2

A comparative sample (comparative cover tube) (2) was produced by the same method as in Comparative Example 1 except that in Comparative Example 1, the base material was changed from SUS to a nylon elastomer (manufactured by Evonik Industries, VESTAMID E47-S1) tube (outer diameter: 2.80 mm).

### Comparative Example 3

A comparative sample (comparative cover tube) (3) was produced by the same method as in Comparative Example 1 except that in Comparative Example 1, the base material was changed from SUS to a polyester elastomer (manufactured by TOYOBO Co., Ltd., PELPRENE E-450B) tube (outer diameter: 2.46 mm).

### Comparative Example 4

A comparative sample (comparative cover tube) (4) was produced by the same method as in Comparative Example 1 except that in Comparative Example 1, the base material was changed from SUS to a polyethylene (manufactured by AS ONE Corporation, Polyethylene tube hose 6-608-02) tube (outer diameter: 4.00 mm).

### Comparative Example 5

4,4′-Diphenylmethane diisocyanate (manufactured by KANTO CHEMICAL CO., INC., product name: methylenediphenyl 4,4′-diisocyanate) was added to N,N-dimethylformamide (DMF) so that the final concentration in the coating liquid was 0.1 mass %, and the mixture was slightly shaken and stirred to be dissolved, and it was visually confirmed that there was no solid undissolved, thereby obtaining a comparative coating liquid (5a).

Next, the block copolymer (1) synthesized in Synthesis Example 1 was dissolved in N, N-dimethylformamide (DMF) so that the final concentration in the coating liquid was 4.5 mass %, and the mixture was stirred for 1 hour using a stirrer. It was visually confirmed that there was no solid undissolved, thereby preparing a comparative coating liquid (5b).

A rod (outer diameter: 3 mm) made of SUS (manufactured by MISUMI Corporation, SUS304 Polishing Rod RGOS3-200) as a base material was immersed in the comparative coating liquid (5a), and the rod was dip-coated (pulling speed: 8 mm/sec). The rod to which the comparative coating liquid (5a) was attached was dried at room temperature (25° C.) for 1 hour to form a comparative coating film (5a) on the surface of the rod (comparative rod (5a)). The comparative rod (5a) has a cover layer (dry film thickness: 0.2 μm) containing 4,4′-diphenylmethane diisocyanate on the surface as the comparative coating film (5a).

Next, the comparative rod (5a) was immersed in the comparative coating liquid (5b), and the rod was dip-coated (pulling speed: 8 mm/sec). The rod to which the comparative coating liquid (5b) was attached was dried at room temperature (25° C.) for 1 hour to remove DMF, and the comparative coating film (5b) was formed on the comparative coating film (5a) (comparative rod (5)). Further, the comparative rod (5) was stored in an oven at 80° C. for 5 hours, and the comparative coating film (5a) and the comparative coating film (5b) were heat-treated (comparative rod (5′)). The comparative rod (5′) was dried at room temperature (25° C.) for 1 hour to produce a comparative sample (comparative cover rod) (5) having a cover layer (surface lubricating layer) (dry film thickness: 1.2 μm) including a crosslinked copolymer derived from the block copolymer (1) on the surface. Note that the surface lubricating layer is the sum of two layers formed by the comparative coating film (5a) and the comparative coating film (5b).

The sliding durability of each of the samples (1) to (4) produced in Example 1 to 4 and the comparative samples (1) to (5) produced in Comparative Examples 1 to 5 was evaluated according to the following methods. The results are shown in Table 1 below.

### Evaluation of Sliding Durability

For each sample, sliding durability (durability of the surface lubricating layer) was evaluated according to the following method.

Each sample was set in a pinch tester (DL1000 manufactured by OAKRIVER TECHNOLOGY) in a state of being immersed in tap water and was slid 50 times at a grip force of 500 gf, a test speed of 8.3 mm/s, and a test stroke of 25 mm (grip pad material: silicone, grip pad height: 12.35 mm).

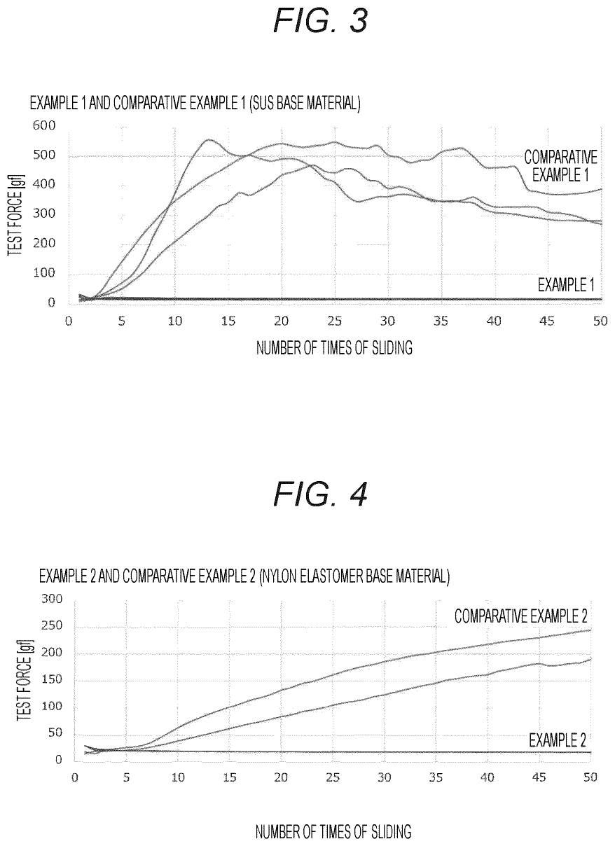

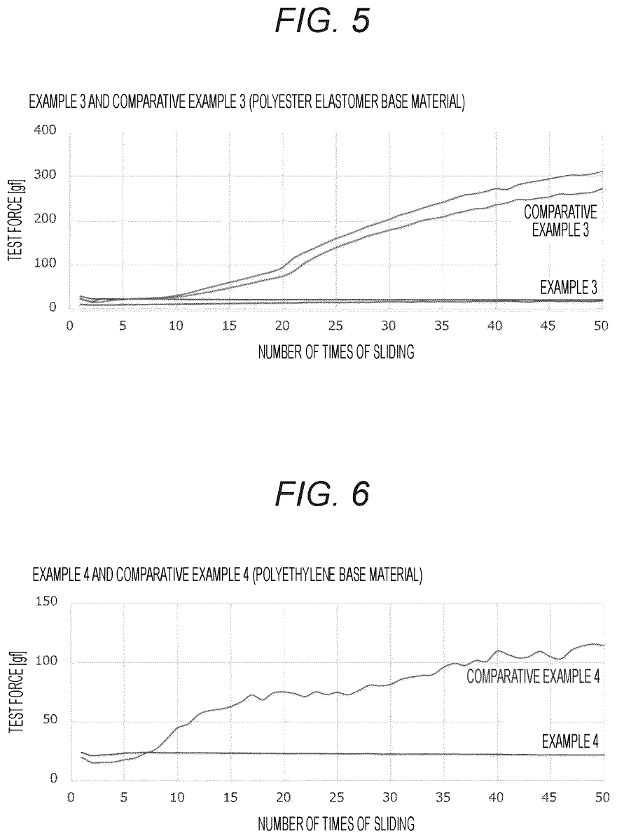

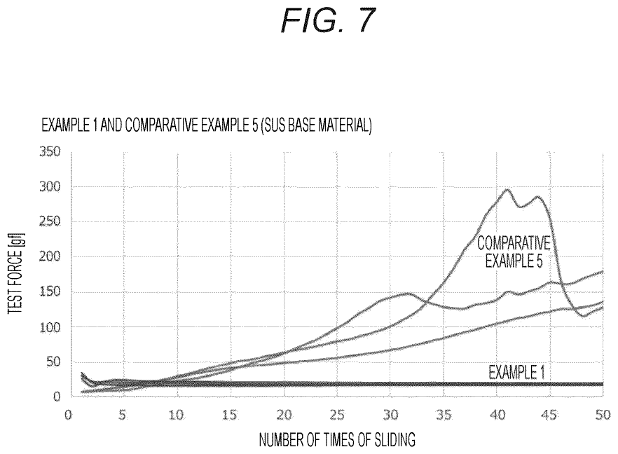

The test force (Tensile Force) (gf) at the time of sliding was measured, and the relationship between the number of times of sliding and the resistance value is shown in FIGS. 3 to 7. FIG. 3 is a graph showing sliding durability against an SUS base material and corresponds to Example 1 and Comparative Example 1. FIG. 4 is a graph showing sliding durability against a nylon elastomer base material and corresponds to Example 2 and Comparative Example 2. FIG. 5 is a graph showing sliding durability against a polyester elastomer base material and corresponds to Example 3 and Comparative Example 3. FIG. 6 is a graph showing sliding durability against a polyethylene base material and corresponds to Example 4 and Comparative Example 4. FIG. 7 is a graph showing sliding durability against an SUS base material and corresponds to Example 1 and Comparative Example 5. Note that, in the sliding measurement of each sample, sliding was performed 50 times, and the average sliding measurements was plotted in the drawing. For each sample, 1 to 3 samples were prepared, and measurement was performed at n=1 to 3 (n plotted lines in the drawing).

Evaluation of test force after sliding 50 times and comprehensive evaluation of sliding durability based on the following criteria were performed. Note that it is determined that the lower the test force is, the more excellent the sliding durability is.

### Comprehensive Evaluation of Sliding Durability

After sliding 50 times in the pinch test,
X . . . test force at 50th cycle is 100 gf or more: with peeling
O . . . test force at 50th cycle is less than 100 gf: without peeling

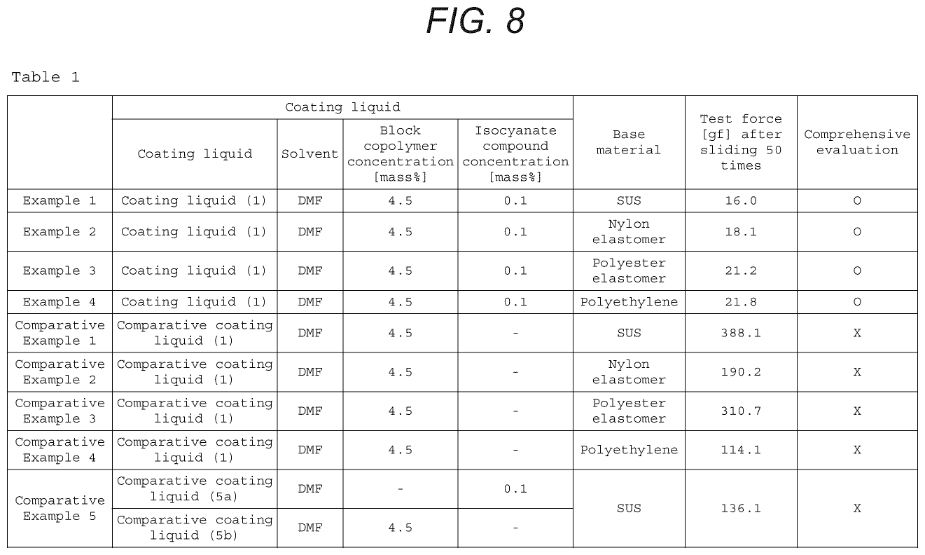

Table 1 (FIG. 8) shows that a medical device having a surface lubricating layer excellent in sliding durability and sliding property can be obtained by applying a coating liquid containing an isocyanate compound and a block copolymer onto a base material layer. This is considered to be because in the coating film formed by the coating liquid, the isocyanate compound and the block copolymer were crosslinked, polymerization was promoted, and a strong surface lubricating layer was formed.

On the other hand, in the case of using a coating liquid containing no isocyanate compound (that is, a coating liquid containing only a block copolymer), the sliding durability of the surface lubricating layer was poor (Comparative Examples 1 to 4). This is considered to be because crosslinking or polymerization of the block copolymer was not promoted, and a strong surface lubricating layer was not formed, so that a part of the surface lubricating layer was peeled off by the time of sliding 50 times.

In Comparative Example 5, an isocyanate compound and a block copolymer were applied with different coating liquids to form a coating film containing the block copolymer on a coating film containing the isocyanate compound. As a result, it was found that the obtained surface lubricating layer had low sliding durability. This is considered to be because crosslinking or polymerization of the block copolymer was not promoted, and a strong surface lubricating layer was not formed, so that a part of the surface lubricating layer was peeled off at the time of sliding 50 times.

The detailed description above describes embodiments of a medical device and a method for manufacturing a medical device. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

### REFERENCE SIGNS LIST

10 Medical device
1 Base material layer
1a Base material layer core portion
1b Base material surface layer
2 Lubricating layer

What is claimed is:

1. A method for manufacturing a medical device, the medical device including a base material layer and a surface lubricating layer supported on at least a part of the base material layer, the method comprising:
preparing a coating liquid containing a block copolymer having a structural unit (A) derived from a reactive monomer having an epoxy group and a structural unit (B) derived from a hydrophilic monomer, an isocyanate compound having two or more isocyanate groups, and a solvent; and
applying the coating liquid onto the base material layer.

2. The method according to claim 1, wherein the coating liquid contains the block copolymer and the isocyanate compound at a mass ratio of 100:0.5 to 100:8.

3. The method according to claim 1, wherein the isocyanate compound is an aliphatic or aromatic isocyanate compound having 6 carbon atoms to 20 carbon atoms.

4. The method according to claim 1, wherein the isocyanate compound comprises at least one selected from the group consisting of hexamethylene diisocyanate, toluene diisocyanate, and 4,4′-diphenylmethane diisocyanate.

5. The method according to claim 1, further comprising applying the coating liquid onto the base material layer to form a coating film, and then heat-treating the coating film at a temperature of 80° C. to 100° C. for 1 hour to 10 hours.

6. The method according to claim 1, wherein the reactive monomer having an epoxy group comprises at least one selected from the group consisting of glycidyl acrylate, glycidyl methacrylate, 3,4-epoxycyclohexyl methyl acrylate, 3,4-epoxycyclohexyl methyl methacrylate, β-methyl glycidyl methacrylate, and allyl glycidyl ether.

7. The method according to claim 1, wherein the hydrophilic monomer comprises at least one selected from the group consisting of N, N-dimethylacrylamide, acrylamide, 2-hydroxyethyl methacrylate, and N-vinylpyrrolidone.

8. The method according to claim 1, wherein the solvent comprises at least one selected from the group consisting of tetrahydrofuran, N, N-dimethylformamide, dimethyl sulfoxide, and acetone.

9. The method according to claim 1, wherein the base material layer comprises at least one selected from the group consisting of a metal, a polyamide resin, a polyolefin resin, a polyester resin, and a polyurethane resin.

10. A medical device comprising:
a base material layer; and
a surface lubricating layer supported on at least a part of the base material layer, wherein the surface lubricating layer comprises a crosslinking reaction product of an epoxy group of a block copolymer having a structural unit (A) derived from a reactive monomer having an epoxy group and a structural unit (B) derived from a hydrophilic monomer and an isocyanate group of an isocyanate compound having two or more isocyanate groups.

11. The medical device according to claim 10, wherein the reactive monomer having an epoxy group comprises at least one selected from the group consisting of glycidyl acrylate, glycidyl methacrylate, 3,4-epoxycyclohexyl methyl acrylate, 3,4-epoxycyclohexyl methyl methacrylate, β-methyl glycidyl methacrylate, and allyl glycidyl ether.

12. The medical device according to claim 10, wherein the hydrophilic monomer comprises at least one selected from the group consisting of N,N-dimethylacrylamide, acrylamide, 2-hydroxyethyl methacrylate, and N-vinylpyrrolidone.

13. The medical device according to claim 10, wherein the isocyanate compound comprises at least one selected from the group consisting of hexamethylene diisocyanate, toluene diisocyanate, and 4,4′-diphenylmethane diisocyanate.

14. The medical device according to claim 10, wherein the base material layer comprises at least one selected from the group consisting of a metal, a polyamide resin, a polyolefin resin, a polyester resin, and a polyurethane resin.

15. A lubricating layer for coating a base material of a medical device, the lubricating layer comprising:
a block copolymer having a structural unit (A) derived from a reactive monomer having an epoxy group and a structural unit (B) derived from a hydrophilic monomer, an isocyanate compound having two or more isocyanate groups, and a solvent.

16. The lubricating layer according to claim 15, wherein the lubricating layer contains the block copolymer and the isocyanate compound at a mass ratio of 100:0.5 to 100:8.

17. The lubricating layer according to claim 15, wherein the isocyanate compound is an aliphatic or aromatic isocyanate compound having 6 carbon atoms to 20 carbon atoms.

18. The lubricating layer according to claim 15, wherein the isocyanate compound comprises at least one selected from the group consisting of hexamethylene diisocyanate, toluene diisocyanate, and 4,4′-diphenylmethane diisocyanate.

19. The lubricating layer according to claim 15, wherein the reactive monomer having an epoxy group comprises at least one selected from the group consisting of glycidyl acrylate, glycidyl methacrylate, 3,4-epoxycyclohexyl methyl acrylate, 3,4-epoxycyclohexyl methyl methacrylate, β-methyl glycidyl methacrylate, and allyl glycidyl ether.

20. The lubricating layer according to claim 15, wherein
the hydrophilic monomer comprises at least one selected from the group consisting of N,N-dimethylacrylamide, acrylamide, 2-hydroxyethyl methacrylate, and N-vinylpyrrolidone; and
the solvent comprises at least one selected from the group consisting of tetrahydrofuran, N,N-dimethylformamide, dimethyl sulfoxide, and acetone.
